(19) United States
(12) Patent Application Publication
(10) Pub. No.: US 2022/0175591 A1
(43) Pub. Date: Jun. 9, 2022

(54) ABSORBENT ARTICLE

(71) Applicant: LIVEDO CORPORATION, Ehime (JP)

(72) Inventor: Riho YOSHIDA, Tokushima (JP)

(73) Assignee: LIVEDO CORPORATION, Ehime (JP)

(21) Appl. No.: 17/437,326

(22) PCT Filed: Feb. 13, 2020
(86) PCT No.: PCT/JP2020/005558

§ 371 Date: Sep. 8, 2021

(30) Foreign Application Priority Data
Mar. 11, 2019 (JP) 2019-044009

(51) Int. Cl. A61F 13/56; B01J 20/22; B01J 20/28; A61F 13/47

(52) U.S. Cl. CPC A61F 13/5611; B01J 20/22; B01J 2220/445; A61F 13/4702; B01J 20/28035

(57) ABSTRACT

An absorbent article (1) comprising a top sheet (2), a back sheet (3) and an absorbent body (11) provided therebetween, wherein; the absorbent body (11) has a high-rigidity part (12) and a low-rigidity part (13); the high-rigidity part (12) and the low-rigidity part (13) extend in the front-rear direction, respectively, and are arranged alternately in the width direction; an anti-slipping member (7) is provided on a non-skin facing side of the back sheet (3); and the anti-slipping member (7) is provided so as to astride a first high-rigidity side part (12A) closest to one end in the width direction of the absorbent body (11) and a second high-rigidity side part (12B) closest to the other end in the width direction of the absorbent body (11), and not to extend outward in the width direction from the first high-rigidity side part (12A) and the second high-rigidity side part (12B).

### TECHNICAL FIELD

The present invention relates to an absorbent article such as an urine pad, an incontinence pad and a sanitary napkin, and more particularly relates to an absorbent article that can be disposed on underwear such as pants and shorts to be used.

### BACKGROUND ART

Conventionally, an absorbent article provided with a hook member or an adhesive layer on a non-skin facing side thereof, that is used by being attached to a skin facing side of underwear such as pants and shorts with the hook member or the adhesive layer, is known (for example, Patent Literatures 1 and 2). Further, an absorbent article provided with an anti-slipping layer on a non-skin facing side thereof is also known (for example, Patent Literature 3).

### CITATION LIST

### Patent Literature

Patent Literature 1
Japanese Unexamined Laid-open Patent Application Publication No. H11-506365

Patent Literature 2
Japanese Unexamined Laid-open Patent Application Publication No. 2003-210524

Patent Literature 3
Japanese Unexamined Laid-open Patent Application Publication No. 2008-11932

### SUMMARY OF INVENTION

### Technical Problem

In the case where an absorbent article is attached to a skin facing side of underwear such as pants or shorts to be used, the absorbent article is held between wearer's thighs and inward force in a width direction is applied to the absorbent article, or shear stress is generated between the absorbent article and the underwear by moving the thighs. As a result, the absorbent article may be distorted or the attached position of the absorbent article with respect to the underwear may be displaced, so that wearing feeling of the absorbent article may be deteriorated.

The present invention has been made in view of the above circumstances, and an object of the present invention is to provide an absorbent article that can be used by being attached to a skin facing side of underwear such as pants and shorts and is less likely to be displaced with respect to underwear and significantly distorted when it is attached to a skin facing side of underwear and is used.

### Solution to Problem

The disposable diaper of the present invention which solves the above problems is an absorbent article having a front-rear direction and a width direction, and comprising a top sheet, a back sheet and an absorbent body provided therebetween, wherein: the absorbent body has a high-rigidity part and a low-rigidity part; the high-rigidity part and the low-rigidity part extend in the front-rear direction, respectively, and are arranged alternately in the width direction; an anti-slipping member is provided on a non-skin facing side of the back sheet; and the anti-slipping member is provided so as to astride a first high-rigidity side part, that is the high-rigidity part closest to one end in the width direction of the absorbent body, and a second high-rigidity side part, that is the high-rigidity part closest to the other end in the width direction of the absorbent body, and not to extend outward in the width direction from the first high-rigidity side part and the second high-rigidity side part.

In the absorbent article of the present invention, since the high-rigidity part and the low-rigidity part are formed in the absorbent body as described above, when the absorbent article is held between both thighs of a wearer and inward force in the width direction is applied to the absorbent article, irregular creases are less likely to be formed in the absorbent body and the inward force in the width direction can be suitably absorbed. And, since the anti-slipping member is provided so as to astride the first high-rigidity side part and the second high-rigidity side part provided on both side in the width direction of the absorbent body, the absorbent article is less likely to be displaced with respect to underwear in placing the absorbent article on a skin facing side of underwear such as pants and shorts. In addition, since the absorbent article is imparted with rigidity in the width direction, when the inward force in the width direction is released from the absorbent article, the absorbent article tends to spread in its original flat shape. Further, since the anti-slipping member is provided so as not to extend outward in the width direction from the first high-rigidity side part and the second high-rigidity side part, slipperiness to the movement of underwear at both ends of the absorbent article in the width direction is increased, and the absorbent article is less likely to be distorted even when a wearer moves thighs significantly. Therefore, when the absorbent article of the present invention is attached to underwear to be worn, the absorbent article is less likely to be displaced with respect to underwear and is less likely to be significantly distorted, resulting in providing an excellent wearing feeling.

The absorbent body may be configured to contain an absorbent polymer but not contain a pulp fiber between sheet members, wherein the absorbent body has a sealed part in which the sheet members are bonded to each other and a non-sealed part in which the sheet members are not bonded to each other, and the absorbent polymer is disposed in the non-sealed part. In this case, the sealed part can form the low-rigidity part and the non-sealed part can form the high-rigidity part. Therefore, the absorbent article of the present invention may be configured that: the absorbent article has a front-rear direction and a width direction, and comprise a top sheet, a back sheet and an absorbent body provided therebetween, wherein: the absorbent body is configured to contain an absorbent polymer but not contain a pulp fiber between sheet members; the absorbent body has a sealed part in which the sheet members are bonded to each other and a non-sealed part in which the sheet members are not bonded to each other, and the absorbent polymer is disposed in the non-sealed part; the sealed part and the non-sealed part extend in the front-rear direction, respectively, and are arranged alternately in the width direction; an anti-slipping member is provided on a non-skin facing side of the back sheet; and the anti-slipping member is provided so as to astride a first non-sealed side part, that is the non-sealed part closest to one end in the width direction of the absorbent body, and a second non-sealed side part, that is the non-sealed part closest to the other end in the width direction of the absorbent body, and not to extend outward in the width direction from the first non-sealed side part and the second non-sealed side part.

It is preferable that a plurality of the anti-slipping members are aligned in the front-rear direction. By providing the anti-slipping member in this manner, shear stress generated in one anti-slipping member is not directly transmitted to another anti-slipping member in the state that the absorbent article is attached to underwear. Therefore, it is possible to prevent the position where the absorbent article is attached to the underwear from displacing and the absorbent article from being distorted over a wide range. Further, by arranging a plurality of the anti-slipping members in the front-rear direction, it becomes easy to bend the absorbent article in the front-rear direction, and fittability of the absorbent article to a wearer's crotch can be improved.

It is preferable that the anti-slipping member is not provided on a front side of a front end of the absorbent body and on a rear side of a rear end of the absorbent body. In the absorbent article, rigidity is particularly low at front and rear end parts thereof where the absorbent body does not exist, and thus, even if the anti-slipping member is provided on these parts, pressing force against the underwear is weak and it is difficult to obtain a desired anti-slipping effect. Rather, when the anti-slipping member is provided at the front and rear end parts of the absorbent article where the absorbent body does not exist, the front and rear end parts of the absorbent article may be folded and caught in underwear in that state, resulting in deteriorating wearing feeling of the absorbent article.

It is preferable that attaching-fixing members made of a hook member or an adhesive layer are provided on the non-skin facing side of the back sheet on a front side and a rear side of the anti-slipping member. Thereby, the absorbent article can be stably attached to underwear. In addition, since the anti-slipping member is provided on the non-skin facing side of the back sheet, an installation area of the attaching-fixing member can be reduced, and when the absorbent article is attached to underwear, damage to fabric of the underwear on the skin facing side can be suppressed.

It is preferable that the attaching-fixing member is provided so as to overlap with a front end of the absorbent body or on a front side of a front end of the absorbent body, or provided so as to overlap with a rear end of the absorbent body or on a rear side of a rear end of the absorbent body. By providing the attaching-fixing member in this manner, the absorbent article can be stably fixed to a skin facing side of underwear.

It is preferable that the attaching-fixing member is wider in the width direction than the anti-slipping member. Thereby, the absorbent article can be stably fixed to a skin facing side of underwear.

It is preferable that the anti-slipping member is formed of a resin layer. Thereby, anti-slipping effect of the anti-slipping member is likely to be suitably exerted.

It is preferable that a fiber sheet is provided between the absorbent body and the back sheet, the fiber sheet has a larger mass per unit area than the top sheet and the back sheet, and the fiber sheet is provided so as to overlap with the entire low-rigidity part or the entire sealed part. By providing the fiber sheet in this manner, the absorbent article easily spreads in a flat shape in combination with the effect of the anti-slipping member, in the state that inward force in the width direction is not applied to the absorbent article.

### Advantageous Effects of Invention

In the absorbent article of the present invention, since the high-rigidity part and the low-rigidity part are formed in the absorbent body, when the absorbent article is held between both thighs of a wearer and inward force in the width direction is applied to the absorbent article, irregular creases are less likely to be formed in the absorbent body and the inward force in the width direction can be suitably absorbed. And, since the anti-slipping member is provided so as to astride the first high-rigidity side part and the second high-rigidity side part provided on both side in the width direction of the absorbent body, the absorbent article is less likely to be displaced with respect to underwear in placing the absorbent article on a skin facing side of underwear. In addition, since the absorbent article is imparted with rigidity in the width direction, when the inward force in the width direction is released from the absorbent article, the absorbent article tends to spread in its original flat shape. Further, since the anti-slipping member is provided so as not to extend outward in the width direction from the first high-rigidity side part and the second high-rigidity side part, slipperiness to the movement of underwear at both ends of the absorbent article in the width direction is increased, and the absorbent article is less likely to be distorted even when a wearer moves thighs significantly. Therefore, when the absorbent article of the present invention is attached to underwear to be worn, the absorbent article is less likely to be displaced with respect to underwear and is less likely to be significantly distorted, resulting in providing an excellent wearing feeling.

### BRIEF DESCRIPTION OF THE DRAWINGS

FIG. 1 shows an example of an absorbent article of the present invention, and shows a plan view of the absorbent article as viewed from a skin facing side thereof.

FIG. 2 shows a plan view of the absorbent article shown in FIG. 1 as viewed from a non-skin facing side thereof.

FIG. 3 shows a cross-sectional view taken along a line of the absorbent article shown in FIGS. 1 and 2.

FIG. 4 shows an example of an absorbent body provided in the absorbent article shown in FIGS. 1 and 2, and shows a cross-sectional view in the width direction of the absorbent body.

FIG. 5 shows a partially cutaway plan view of the absorbent body shown in FIG. 4 as viewed from a skin facing side thereof.

FIG. 6 shows a cross-sectional view in the width direction of the absorbent article provided with the absorbent body shown in FIGS. 4 and 5.

### DESCRIPTION OF THE PREFERRED EMBODIMENTS

The absorbent article of the present invention is explained with reference to drawings. However, the present invention is not limited to the embodiments shown in the drawings.

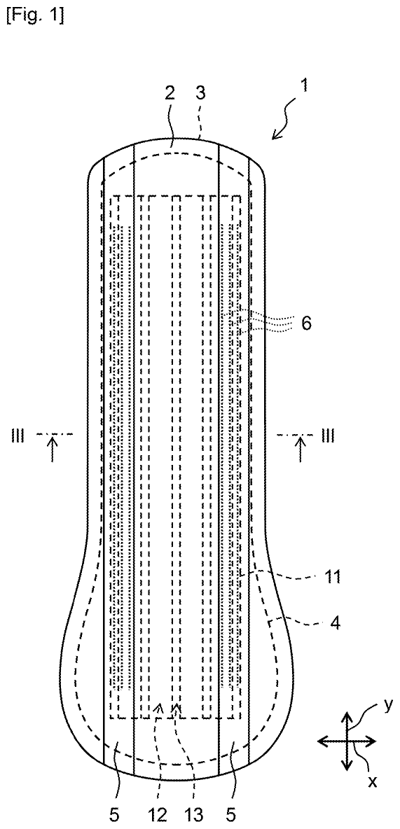

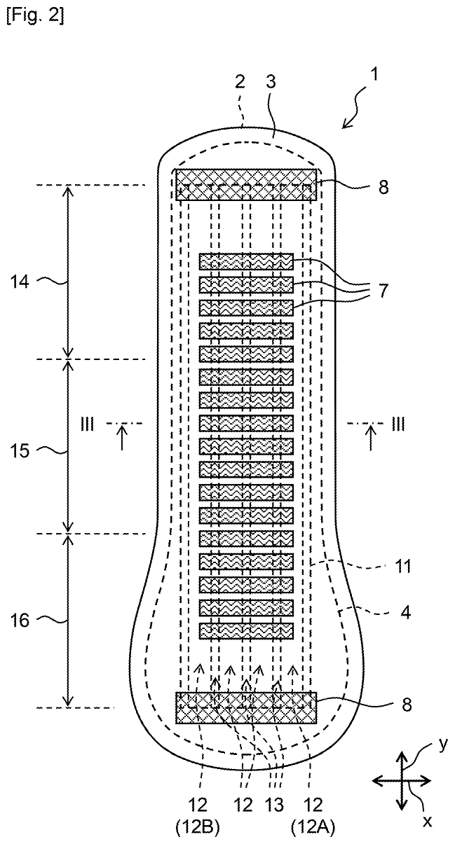

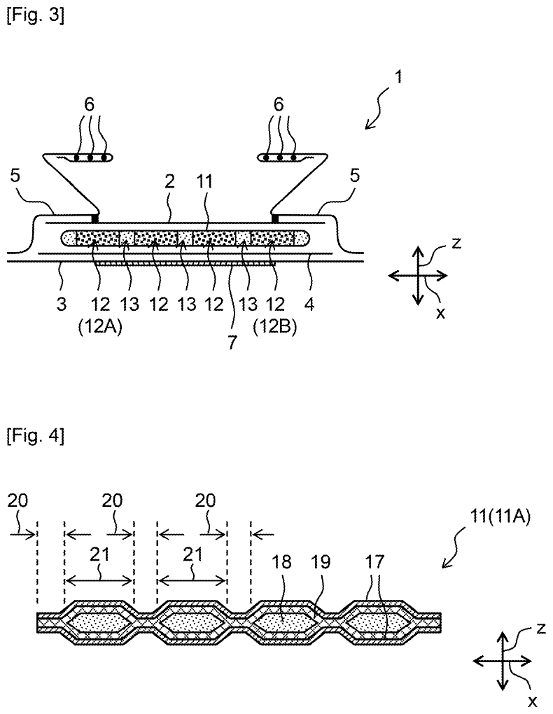

FIGS. 1 to 3 show a configuration example of an incontinence pad as an example of an absorbent article of the present invention. FIG. 1 shows a plan view of an absorbent article (an incontinence pad) as viewed from a skin facing side thereof, FIG. 2 shows a plan view of the absorbent article shown in FIG. 1 as viewed from a non-skin facing side thereof, and FIG. 3 shows a cross-sectional view taken along a line III-III of the absorbent article shown in FIGS. 1 and 2. In the drawings of the present application, an arrow x represents a width direction, an arrow y represents a front-rear direction, and a direction perpendicular to a plane formed by the arrows x and y represents a thickness direction z. In FIGS. 1 and 2, an upper side of the drawing corresponds to a front side of the absorbent article, and a lower side of the drawing corresponds to a rear side of the absorbent article.

An absorbent article 1 comprises a top sheet 2, a back sheet 3 and an absorbent body 11 provided therebetween. The top sheet 2 is disposed on a skin facing side of the absorbent body 11, and the back sheet 3 is disposed on a non-skin facing side of the absorbent body 11. Excrement that has passed through the top sheet 2 is stored by the absorbent body 11. The back sheet 3 prevents excrement from leaking to the outside.

The absorbent article 1 has a front-rear direction y and a width direction x. The front-rear direction y means a direction extending in a front-rear direction at a crotch of a wearer when the absorbent article is worn. The width direction x means a direction orthogonal to the front-rear direction y on the same plane as the absorbent article, and corresponds to a left-right direction of a wearer when the absorbent article is worn. In addition, a skin facing side means a side facing a wearer's skin and a non-skin facing side means an opposite side of that when a wearer wears the absorbent article.

The top sheet 2 is disposed on a skin facing side of the absorbent body 11 and is provided so as to face a wearer's skin when the absorbent article is worn. The top sheet 2 is preferably liquid permeable. As the top sheet 2, a nonwoven fabric made from hydrophilic fibers such as cellulose, rayon and cotton; a nonwoven fabric which is formed from hydrophobic fibers such as polyolefin (e.g., polypropylene and polyethylene), polyester (e.g., PET) and polyamide (e.g., nylon), and in which the hydrophobic fibers are hydrophilized with a surfactant on the surface thereof; or the like can be used, for example. As the top sheet 2, a woven fabric, a knitted fabric, a plastic film having holes may be also used.

The back sheet 3 is disposed on a non-skin facing side of the absorbent body 11 and is preferably liquid impermeable. As the back sheet 3, a nonwoven fabric made from hydrophobic fibers such as polyolefin (e.g., polypropylene and polyethylene), polyester (e.g., PET) and polyamide (e.g., nylon); a plastic film; or the like can be used, for example. As the back sheet 3, a laminate of a nonwoven fabric and a plastic film may be used. A non-skin facing side of the back sheet 3 serves as an underwear facing surface when the absorbent article is attached to underwear such as pants or shorts.

It is preferable that the absorbent article 1 is provided with side sheets 5 extending in the front-rear direction y on both sides of the top sheet 2 in the width direction x. The side sheets 5 are joined to both sides of the top sheet 2 in the width direction x. In FIGS. 1 and 3, three rising elastic members 6 are disposed on each of the side sheets 5 provided on one side and the other side in the width direction x. In using the absorbent article, an inner portion of the side sheet 5 rises toward a wearer's skin due to contraction force of the rising elastic member 6 to form a rising flap, thereby preventing lateral leakage of excrement such as urine. The side sheet 5 can be made of a liquid-impermeable plastic film, a liquid-impermeable nonwoven fabric or the like, and a sheet material that can be used for the back sheet 3 can be employed.

In the drawings, a fiber sheet 4 is provided between the absorbent body 11 and the back sheet 3, and the fiber sheet 4 can improve shape-retaining property of the absorbent article 1. The fiber sheet 4 can be made of crepe paper or a nonwoven fabric.

In the case where a nonwoven fabric is used for the top sheet 2, the back sheet 3 or the side sheet 5, a spunbonded nonwoven fabric, an air-through nonwoven fabric, a point-bonded nonwoven fabric, a meltblown nonwoven fabric, an airlaid nonwoven fabric, an SMS nonwoven fabric or the like is preferably used as the nonwoven fabric.

The absorbent body 11 is not particularly limited as long as it contains an absorbent material that is able to absorb excrement such as urine. As the absorbent body 11, a shaped product of an absorbent material, which is formed into a certain shape, may be used, or the shaped product wrapped with a cover sheet such as paper (e.g., tissue paper and thin paper) and a liquid-permeable nonwoven fabric may be used. Examples of the absorbent material include, for example, a hydrophilic fiber such as a cellulose fiber, and an absorbent polymer such as a polyacrylic absorbent polymer, a polyasparaginic absorbent polymer, a cellulosic absorbent polymer and a stark-acrylonitrile absorbent polymer. The absorbent material may include a thermal fusion fiber such as a polyolefin (e.g., polyethylene and polypropylene) fiber, a polyester (e.g., PET) fiber and a polyamide fiber. These thermal fusion fibers may be hydrophilized with a surfactant or the like to increase affinity with urine or the like.

The absorbent material preferably includes a hydrophilic fiber in view of increasing absorption rate of urine or the like. In addition, in view of enhancing absorption capacity, the absorbent material preferably includes an absorbent polymer. Therefore, the absorbent body 11 preferably contains both a hydrophilic fiber (especially a pulp fiber) and an absorbent polymer. In this case, the absorbent material to be used is preferably formed by mixing an absorbent polymer with a hydrophilic fiber assembly, or dispersing an absorbent polymer on a hydrophilic fiber assembly, for example.

A shape (planar shape) of the absorbent body 11 is not particularly limited. The shape of the absorbent body 11 may be determined as appropriate according to application, and examples of that include a substantially rectangular shape, an hourglass shape, a center nipped-in gourd shape, a battledore shape and others.

The absorbent body 11 has a high-rigidity part 12 and a low-rigidity part 13. The high-rigidity part 12 is formed to have a relatively higher rigidity than the low-rigidity part 13. The high-rigidity part 12 and the low-rigidity part 13 can be formed as follows, for example. (1) Mass per unit area of the high-rigidity part 12 is made larger than that of the low-rigidity part 13. (2) The absorbent body 11 comprises fiber aggregate as a main component, and the fiber aggregate is compressed at the high-rigidity part 12. (3) The absorbent body 11 is configured to contain an absorbent polymer but not contain a pulp fiber between sheet members, and an amount of the absorbent polymer per unit area in the high-rigidity part 12 is made larger than that in the low-rigidity part 13.

The high-rigidity part 12 and the low-rigidity part 13 extend in the front-rear direction y, respectively, and the high-rigidity part 12 and the low-rigidity part 13 are arranged alternately in the width direction x in the absorbent body 11. That is, the high-rigidity part 12 and the low-rigidity part 13 are arranged in a stripe shape extending in the front-rear direction y in the absorbent body 11. As the absorbent body 11 is configured in this manner, when the absorbent article 1 is held between both thighs of a wearer and inward force in the width direction x is applied to the absorbent article 1, irregular creases are less likely to be formed in the absorbent body 11 and the inward force in the width direction x can be suitably absorbed. For example, when the inward force in the width direction x is applied to the absorbent article 1, the absorbent body 11 can be bent at the low-rigidity part 13 to be narrowed in the width direction x. Meanwhile, even when such a force is applied to the absorbent body 11, the high-rigidity part 12 of the absorbent body 11 is suppressed from being deformed and flatness of the absorbent body 11 as a whole tends to be maintained. Therefore, the absorbent body 11 is less likely to be largely distorted, wearing feeling of the absorbent article is improved, and urine or the like excreted from a wearer is easily suitably absorbed by the absorbent body 11.

A plurality of the high-rigidity parts 12 are arranged side by side in the width direction x, and the high-rigidity part 12 closest to one end in the width direction x of the absorbent body 11 is referred to as a first high-rigidity side part 12A, and the high-rigidity part 12 closest to the other end in the width direction x of the absorbent body 11 is referred to as a second high-rigidity side part 12B. Three or more high-rigidity parts 12 are preferably provided side by side in the width direction x, and in this case, one high-rigidity part 12 is provided between the first high-rigidity side part 12A and the second high-rigidity side part 12B, or two or more high-rigidity part 12 are provided side by side in the width direction x between them. in addition, preferably eight or less high-rigidity parts 12, more preferably six or less high-rigidity parts 12 are provided side by side in the width direction x. It is also preferable that a plurality of the low-rigidity parts 13 are provided side by side in the width direction x, more preferably 3 or more those, and preferably 9 or less those, more preferably 7 or less those are provided side by side in the width direction x. It is preferable that the high-rigidity part 12 and the low-rigidity part 13 extend over the entire of the absorbent body 11 in the front-rear direction y, respectively.

A length of each of the high-rigidity part 12 and the low-rigidity part 13 in the width direction x is not particularly limited, however, it is preferable that the length of the high-rigidity part 12 in the width direction x is longer than the length of the low-rigidity part 13 in the width direction x. Specifically, it is preferable that all the high-rigidity parts 12 have a longer length in the width direction x than the low-rigidity parts 13. Thereby, shape-retaining property of the absorbent body 11 in using the absorbent article can be improved, and the absorbent body 11 is likely to be formed in a flat shape in the state that the inward force in the width direction x is not applied to the absorbent body 11. A ratio of the length of the high-rigidity part 12 to the length of the low-rigidity part 13 in the width direction x is preferably more than 50/50, more preferably 60/40 or more, and preferably 95/5 or less, more preferably 90/10 or less, even more preferably 85/15 or less, as a ratio of mean value of the length of the high-rigidity part 12 in the width direction x/mean value of the length of the low-rigidity part 13 in the width direction x.

The absorbent article 1 is provided with an anti-slipping member 7 on a non-skin facing side of the back sheet 3. By providing the anti-slipping member 7 on the non-skin facing side of the back sheet 3, when the absorbent article 1 is placed on the skin facing side of underwear such as pants and shorts, the absorbent article 1 is less likely to be displaced with respect to the underwear. Further, in the case where an attaching-fixing member 8 such as a hook member or an adhesive layer is provided on the non-skin facing side of the back sheet 3, an installation area of the attaching-fixing member 8 can be reduced, whereby damage to fabric of the underwear on its skin facing side can be suppressed when the absorbent article 1 is attached to the underwear.

The anti-slipping member 7 is provided so as to astride the first high-rigidity side part 12A and the second high-rigidity side part 12B. As explained above, the absorbent body 11 is likely to be bent at the low-rigidity part 13 when the inward force in the width direction x is applied to the absorbent article 1. However, by providing the anti-slipping member 7 so as to astride the first high-rigidity side part 12A and the second high-rigidity side part 12B, the anti-slipping member 7 is likely to maintain contact with underwear at the high-rigidity part 12, whereby the anti-slipping member 7 is easily kept at the initial placed position of the underwear. Further, by providing the anti-slipping member 7 in this manner, the absorbent article 1 is imparted with rigidity in the width direction x in the range from the first high-rigidity side part 12A to the second high-rigidity side part 12B. Therefore, when the inward force in the width direction x is released from the absorbent article 1, the absorbent article 1 tends to spread in its original flat shape.

Meanwhile, the anti-slipping member 7 is provided so as not to extend outward in the width direction x from the first high-rigidity side part 12A and the second high-rigidity side part 12B. By providing the anti-slipping member 7 in this manner, slipperiness to the movement of underwear at both ends of the absorbent article 1 in the width direction x is increased when the absorbent article 1 is attached to the underwear. Therefore, even when a wearer moves thighs significantly in the state that the absorbent article 1 is attached to the underwear, the absorbent article 1 is less likely to be distorted, and the absorbent article 1 easily remains to be fixed at a predetermined position of the underwear.

It is preferable that a plurality of the anti-slipping members 7 are aligned in the front-rear direction y. By providing the anti-slipping member 7 in this manner, shear stress generated in one anti-slipping member 7 is not directly transmitted to another anti-slipping member 7 in the state that the absorbent article 1 is attached to underwear. Therefore, the position where the absorbent article 1 is attached to underwear can be prevented from displacing and the absorbent article 1 can be prevented from being distorted over a wide range. Further, by arranging a plurality of the anti-slipping members 7 in the front-rear direction y, it becomes easy to bend the absorbent article 1 in the front-rear direction y, and fittability of the absorbent article 1 to a wearer's crotch can be improved.

Preferably four or more anti-slipping members 7, more preferably eight or more those, even more preferably twelve or more those, are aligned in the front-rear direction y. The upper limit of the number of the anti-slipping member 7 is not particularly limited, and for example, 80 or less or 60 or less anti-slipping members 7 may be aligned in the front-rear direction y. In the case where a plurality of the anti-slipping members 7 are aligned in the front-rear direction y, ½ or more of the total number of anti-slipping members 7 aligned in the front-rear direction y are preferably provided so as to astride the first high-rigidity side part 12A and the second high-rigidity side part 12B, more preferably ¾ or more of those are provided as such, and even more preferably all of those are provided as such. In addition, ½ or more of the total number of anti-slipping members 7 aligned in the front-rear direction y are preferably provided so as not to extend outward in the width direction x from the first high-rigidity side part 12A and the second high-rigidity side part 12B, more preferably ¾ or more of those are provided as such, and even more preferably all of those are provided as such.

The length of each of the anti-slipping member 7 in the front-rear direction y is preferably 1 mm or longer, more, more preferably 3 mm or longer, even more preferably 5 mm or longer, in view of enhancing anti-slipping effect. Meanwhile, the length of each of the anti-slipping member 7 in the front-rear direction y is preferably 30 mm or shorter, more preferably 25 mm or shorter, even more preferably 20 mm or shorter, in view of facilitating smoothly bending of the absorbent article 1 in the front-rear direction y.

Provided that the absorbent body 11 is divided into three equal parts, that is, a front part 14, a rear part 16 and a middle part 15 positioned therebetween, in the front-rear direction y, it is preferable that the anti-slipping member 7 is provided at least at a position overlapping with the middle part 15. For example, the anti-slipping member 7 may be provided only at a position overlapping with the middle part 15 of the absorbent body 11, or may be provided at positions overlapping with the front part 14, the middle part 15 and the rear part 16 of the absorbent body 11, respectively. When the anti-slipping members 7 are provided on the front part 14, the middle part 15 and the rear side part 16 of the absorbent body 11, respectively, anti-slipping effect of the absorbent article 1 can be enhanced. On the other hand, when the anti-slipping member 7 is provided only at the middle part 15 of the absorbent body 11, fittability of the absorbent article 1 is easily enhanced due to that the absorbent article 1 is formed relatively flexible at the front part 14 and the rear part 16, while anti-slipping effect of the absorbent article 1 is ensured.

It is preferable that the anti-slipping member 7 is not provided on a front side of a front end of the absorbent body 11 and on a rear side of a rear end of the absorbent body 11. In the absorbent article 1, rigidity is particularly low at front and rear end parts where the absorbent body 11 does not exist, and thus, even if the anti-slipping member 7 is provided on these parts, pressing force against underwear is weak and it is difficult to obtain a desired anti-slipping effect. Rather, when the anti-slipping member 7 is provided at the front and rear end parts of the absorbent article 1 where the absorbent body 11 does not exist, the front and rear end parts of the absorbent article 1 may be folded and caught in underwear in the folded state, resulting in deteriorating wearing feeling of the absorbent article 1.

The anti-slipping member 7 can be formed of a resin layer. The resin layer preferably has elasticity (specifically, elasticity exerted by force that can be handled by a human hand), and is preferably composed of an elastomer resin. Examples of the elastomer resin include urethane-based resin, silicone-based resin, styrene-based resin (e.g., styrene-butadiene copolymer (SBR), styrene-isoprene-styrene block copolymer (SIS), styrene-butadiene-styrene block copolymer (SBS), styrene-ethylene-butadiene-styrene block copolymer (SEBS), styrene-ethylene-propylene-styrene block copolymer (SEPS) and the like), acrylic resin, α-olefin resin, butadiene resin, vinyl chloride resin, vinyl acetate resin, natural rubber and others.

The resin layer is preferably substantially non-adhesive, and for example, the content of a tackifier component is preferably 5% by mass or less, more preferably 3% by mass or less, even more preferably 1% by mass or less. Adhesives usually contain 10% by mass or more of a tackifier component to exhibit adhesiveness and tackiness, whereas the resin layer of the anti-slipping member 7 preferably does not contains a large amount of such a tackifier component. Examples of the tackifier component include rosin resins, terpene resins, dicyclopentadiene resins, C5 or C9 hydrocarbon resins, alicyclic hydrocarbon resins and others.

It is preferable that the anti-slipping member 7 is formed so as to rise from a surface of the non-skin facing side of the back sheet 3. The height of the anti-slipping member 7 (height from the surface of the non-skin facing side of the back sheet 3) is, for example, preferably 0.1 mm or higher, more preferably 0.2 mm or higher, even more preferably 0.3 mm or higher, and preferably 3.0 mm or lower, more preferably 2.0 mm or lower, even more preferably 1.0 mm or lower.

It is preferable that the anti-slipping member 7 has a durometer hardness A of 90 or less, more preferably 80 or less, even more preferably 70 or less, and preferably 20 or more, more preferably 25 or more, even more preferably 30 or more. Thereby, anti-slipping effect of the anti-slipping member 7 is easily exerted appropriately. The durometer hardness A is measured according to JIS K 6253-3 (2012).

Peeling adhesive strength of the anti-slipping member 7 is preferably less than 0.1 N, more preferably less than 0.05 N, and even more preferably less than 0.03 N. The peeling adhesive strength of the anti-slipping member 7 is measured according to the following method. In accordance with JIS K 6854-2 (1999) “Adhesives—Determination of peel strength of bonded assemblies (180 degree peeling)”, an integrated average load of peeling force is measured, and the average value of 10 tests is used as the peeling adhesive strength. The peeling speed (gripper movement speed) is set 300 mm/min. A test piece used for the measurement is prepared by cutting out a portion of the back sheet where the anti-slipping member is provided (a plurality of the anti-slipping members may be in alignment) in a size of 100 mm×10 mm, sticking the back sheet onto a base substrate having a size of 150 mm×25 mm, and overlaying Kanakin No. 3 cotton cloth of the same size as the base substrate on this. For the cut out back sheet, the surface opposite to the anti-slipping member is stuck onto the base substrate, and the Kanakin No. 3 cotton cloth is overlaid on the surface provided with the anti-slipping member. For the Kanakin No. 3 cotton cloth, one used for JIS L 0803 “Dyeing fastness test” is used. In the case where the size of the portion of the back sheet where the anti-slipping member is provided is less than 100 mm×10 mm, a plurality of the back sheets are joined together to form a size of 100 mm×10 mm. The back sheet provided with the anti-slipping member is stuck onto the Kanakin No. 3 cotton cloth by reciprocating a roller (manufactured by Tester Sangyo Co., Ltd., SA-1003-B, load 2 kgf, width 45 mm, diameter 95 mm) three time to prepare the test piece.

It is preferable that an attaching-fixing member 8 made of a hook member or an adhesive layer is provided on the non-skin facing side of the back sheet 3. By providing the attaching-fixing member 8 on the non-skin facing side of the back sheet 3, the absorbent article 1 can be stably attached to underwear.

The attaching-fixing members 8 are preferably provided on a front side and a rear side of the anti-slipping member 7, whereby the position of the absorbent article 1 on a skin facing side of underwear can be stably fixed while the installation area of the attaching-fixing member 8 is reduced. Further, by reducing the installation area of the attaching-fixing member 8, damage to fabric of the underwear on the skin facing side can be suppressed when the absorbent article 1 is attached to the underwear. The anti-slipping member 7 is preferably provided only between the attaching-fixing member 8 on the front side and the attaching-fixing member 8 on the rear side.

The attaching-fixing member 8 is preferably provided so as to overlap with the front part 14 of the absorbent body 11 or on a front side of the front part 14 of the absorbent body 11, or preferably provided so as to overlap with the rear part 16 of the absorbent body 11 or on a rear side of the rear part 16 of the absorbent body 11. More preferably, the attaching-fixing member 8 is provided so as to overlap with a front end of the absorbent body 11 or on a front side of the front end of the absorbent body 11, or provided so as to overlap with a rear end of the absorbent body 11 or on a rear side of the rear end of the absorbent body 11. By providing the attaching-fixing member 8 in this manner, the absorbent article 1 can be stably fixed to a skin facing side of underwear. From the viewpoint of fixing the absorbent article 1 to the skin facing side of the underwear more stably, it is preferable that the attaching-fixing members 8 are provided so as to overlap with the front end of the absorbent body 11 and overlap with the rear end of the absorbent body 11.

It is preferable that the attaching-fixing member 8 is wider in the width direction x than the anti-slipping member 7. Thereby, the absorbent article 1 can be stably fixed to a skin facing side of underwear. More preferably, the attaching-fixing member 8 is provided so as to extend outward in the width direction x from the first high-rigidity side part 12A and the second high-rigidity side part 12B of the absorbent body 11, and even more preferably, the attaching-fixing member 8 is provided wider in the width direction x than the absorbent body 11.

On the front side of the anti-slipping member 7, only one attaching-fixing member 8 may be provided, or a plurality of the attaching-fixing members 8 may be provided. On the rear side of the anti-slipping member 7, only one attaching-fixing member 8 may be provided, or a plurality of the attaching-fixing members 8 may be provided. For example, a plurality of the attaching-fixing members 8 may be aligned in the front-rear direction y on the front side of the anti-slipping member 7, or a plurality of the attaching-fixing members 8 may be aligned in the front-rear direction y on the rear side of the anti-slipping member 7.

The attaching-fixing member 8 provided on the front side (or rear side) of the anti-slipping member 7 preferably has a length in the front-rear direction y of 5 mm or longer, more preferably 10 mm or longer, and preferably 80 mm or shorter, more preferably 50 mm or shorter, even more preferably 30 mm or shorter. In the case where a plurality of the attaching-fixing members 8 provided on the front side (or rear side) of the anti-slipping member 7 are aligned in the front-rear direction y, it is preferable that the length of the entire attaching-fixing members 8 aligned in the front-rear direction y is in such a range. By providing the attaching-fixing member 8 in this manner, the position of the absorbent article 1 can be stably fixed on a skin facing side of underwear while reducing the installation area of the attaching-fixing member 8.

As the hook member used for the attaching-fixing member 8, a hook member of a hook-and-loop fastener can be employed, and a hook member provide with engaging parts having a shape such as a hook shape, an anchor shape or a mushroom shape is generally well known. The shape of the engaging part of the hook member is not limited to those, and the shape is not particularly limited as long as it can engage with a skin facing side of underwear.

The adhesive layer used for the attaching-fixing member 8 can be formed from a known adhesive, and a type thereof is not particularly limited. The adhesive layer preferably contains a tackifier component in an amount of 10% by mass or more, more preferably 15% by mass or more, and even more preferably 20% by mass or more. The adhesive layer has a peeling adhesive strength, that is described above, of 0.2 N or more, more preferably 0.3 N or more, and even more preferably 0.4 N or more.

From the viewpoint of enhancing shape-retaining property of the absorbent article 1, it is preferable that a fiber sheet 4 is provided between the absorbent body 11 and the back sheet 3. The fiber sheet 4 preferably has a larger mass per unit area than the top sheet 2 and the back sheet 3, whereby rigidity of the portion of the absorbent article 1 where the absorbent body 11 does not exist or rigidity of the low-rigidity part 13 can be increased. The fiber sheet 4 is preferably provided so as to overlap with the entire low-rigidity part 13, and by providing the fiber sheet 4 in this manner, the absorbent article 1 easily spreads in a flat shape in combination with the effect of the anti-slipping member 7, in the state where the inward force in the width direction x is not applied to the absorbent article 1. Mass per unit area of the fiber sheet 4 is preferably, for example, 30 g/m² to 60 g/m².

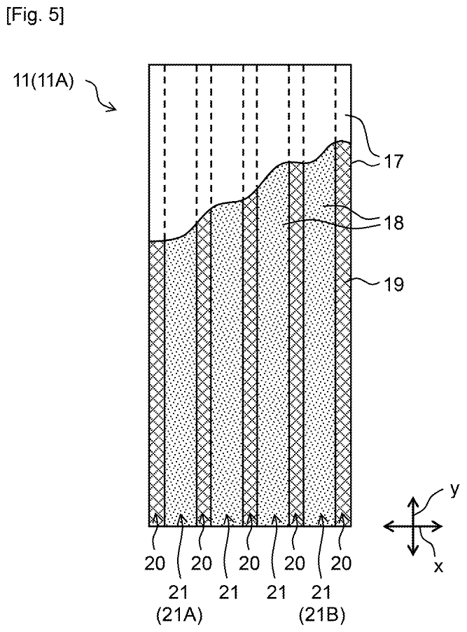

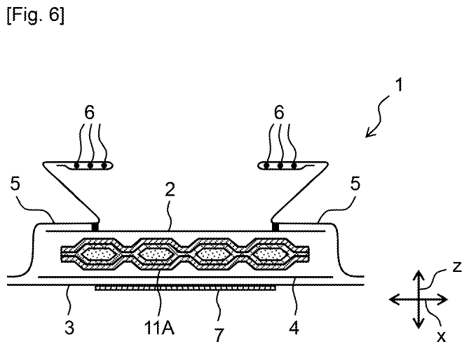

The absorbent body 11 may be a sheet-like absorbent body that is configured to contain an absorbent polymer but not contain a pulp fiber between nonwoven fabrics. This will be explained with reference to FIGS. 4 to 6. FIGS. 4 and 5 show an example of a sheet-like absorbent body, FIG. 4 shows a cross-sectional view in the width direction of the absorbent body, FIG. 5 shows a partially cutaway plan view of the absorbent body shown in FIG. 4, and FIG. 6 shows a cross-sectional view in the width direction of the absorbent article provided with the absorbent body shown in FIGS. 4 and 5. In FIGS. 4 to 6, the sheet-like absorbent body is designated by reference numeral 11A.

The absorbent body 11A comprises a plurality of layers of a sheet member 17, and an absorbent polymer 18 is disposed between the sheet members 17, but a pulp fiber is not disposed therebetween. That is, in the absorbent body 11A, the sheet members 17 are disposed on a skin facing side and a non-skin facing side of the absorbent body 11A, respectively, and an absorbent polymer 18 is disposed between the sheet members 17, but a pulp fiber is not disposed between the sheet members 17. It is noted that the presence of pulp fibers inevitably mixed in the sheet members 17 in the manufacturing process is allowed. As the absorbent body 11A is configured in this manner, the absorbent body 11A can be formed to be thin while having a high absorption capacity.

The sheet member 17 is liquid-permeable, and a sheet member that can be used for the top sheet 2 can be employed. As the sheet member 17, a sheet member composed of a nonwoven fabric, that is, a nonwoven fabric sheet is preferably used. For the sheet member 17, a sheet member on the skin facing side and a sheet member on the non-skin facing side may be provided separately, or one sheet member is folded back, and one side of the fold may serve as a sheet member on the skin facing side and the other side of the fold may serve as a sheet member on the non-skin facing side.

The absorbent body 11A has a sealed part 20 in which the sheet members 17 are bonded to each other and a non-sealed part 21 in which the sheet members 17 are not bonded to each other, and the absorbent polymer 18 is disposed in the non-sealed part 21. The sealed part 20 is formed by joining the sheet members 17 to each other with an adhesive or welding (heat sealing, ultrasonic welding, or the like). The non-sealed part 21 is defined as a part where the sheet members 17 are not joined to each other, and a part other than the sealed part 20 corresponds to the non-sealed part 21. An absorbent polymer may not be disposed in the sealed part 20, however, a small amount of an absorbent polymer may be present therein.

In the absorbent body 11A, the sealed part 20 and the non-sealed part 21 extend in the front-rear direction y, respectively, and the sealed part 20 and the non-sealed part 21 are arranged alternately in the width direction x. In the absorbent body 11A formed in this manner, since the absorbent polymer 18 is basically disposed in the non-sealed part 21, rigidity of the non-sealed part 21 comes to be higher than rigidity of the sealed part 20. As a result, the non-sealed part 21 can function as the high-rigidity part, and the sealed part 20 can function as the low-rigidity part. Therefore, in this case, provided that the non-sealed part 21 closest to one end in the width direction x of the absorbent body 11A is referred to as the first non-sealed side part 21A and the non-sealed part 21 closest to the other end in the width direction x of the absorbent body 11A is referred to as the second non-sealed side part 21B, the anti-slipping member is preferably provided so as to astride the first non-sealed side part 21A and the second non-sealed side part 21B and preferably provided so as not to extend outward in the width direction x from the first non-sealed side part 21A and the second non-sealed side part 21B.

Details of the arrangement and others of the sealed part and the non-sealed part and details of the relationship between the sealed part, the non-sealed part, the anti-slipping member and the attaching-fixing member are referred to by replacing “high-rigidity part” with “non-sealed part” and “low-rigidity part” with “sealed part” in the above description.

In FIGS. 4 and 5, an adhesive is applied to the sheet member 17 to form an adhesive layer 19, and the absorbent polymer 18 is fixed to the sheet member 17 by the adhesive layer 19 in the non-sealed part 21. The adhesive layer 19 may be provided on at least one of the sheet member 17 on the skin facing side and the sheet member 17 on the non-skin facing side, and the adhesive layer 19 is preferably provided on both sheet members 17. At least a part of the absorbent polymer 18 may be fixed to the adhesive layer 19. When the absorbent polymer 18 is fixed to the sheet member 17 by the adhesive layer 19, the absorbent polymer 18 hardly moves between the sheet members 17 before the absorbent polymer 18 absorbs, and the ability of the absorbent body 11A to absorb urine or the like is easily secured. Further, even after the absorbent polymer 18 has absorbed, gelled absorbent polymer 18 is less likely to move between the sheet members 17, and as a result, it hardly occurs to give a wearer uncomfortable feeling due to the lump-formed absorbent polymer 18.

It is preferable that the adhesive layer 19 does not inhibit water absorption or swelling of the absorbent polymer 18 while fixing the absorbent polymer 18. In order to form such an adhesive layer 19, the adhesive layer 19 is preferably formed by a curtain spray method, a spiral coating method, an omega coating method, a coater method or the like.

It is preferable that the sealed part 20 maintains the bonding between the sheet members 17 even when the absorbent polymer 18 absorbs. As a result, after the absorbent polymer 18 has absorbed, the ability of the absorbent body 11A to absorb urine or the like is likely to be secured. From this point of view, the sealed part 20 is preferably formed by bonding the sheet members 17 to each other with an adhesive such as a rubber adhesive or a styrene-based elastomer or formed by welding the sheet members 17 to each other.

The length of each of the sealed parts 20 in the width direction x is preferably 1 mm or longer, more preferably 2 mm or longer, and preferably 10 mm or shorter, more preferably 8 mm or shorter. The length of each of the non-sealed parts 21 in the width direction x is preferably 5 mm or longer, more preferably 8 mm or longer, and preferably 50 mm or shorter, more preferably 30 mm or shorter, even more preferably 20 mm or shorter.

The amount of the absorbent polymer 18 disposed in the non-sealed part 21 is not particularly limited, and is preferably 100 g/m² or more, more preferably 150 g/m² or more, and preferably 400 g/m² or less, more preferably 385 g/m² or less. When the absorbent polymer 18 is disposed in the non-sealed part 21 in an amount of 100 g/m² or more, absorption capacity of the absorbent body 11A can be easily increased and rigidity of the non-sealed part 21 can be easily enhanced. Meanwhile, when the absorbent polymer 18 is disposed in the non-sealed part 21 in an amount of 400 g/m² or less, the bonding between the sheet members 17 in the sealed part 20 is easily maintained even when the absorbent polymer 18 absorbs. The amount of an absorbent polymer in the sealed part 20 is smaller than the amount of an absorbent polymer in the non-sealed part 21.

This application claims priority to Japanese Patent Application No. 2019-044009, filed on Mar. 11, 2019. All of the contents of the Japanese Patent Application No. 2019-044009, filed on Mar. 11, 2019, are incorporated by reference herein.

### REFERENCE SIGNS LIST

1: an absorbent article
2: a top sheet
3: a back sheet
4: a fiber sheet
5: a side sheet
6: a rising elastic member
7: an anti-slipping member
8: an attaching-fixing member
11, 11A: an absorbent body
12: a high-rigidity part, 12A: a first high-rigidity side part, 12B: a second high-rigidity side part
13: a low-rigidity part
14: a front part
15: a middle part
16: a rear part
17: a sheet member
18: an absorbent polymer
19: an adhesive layer
20: a sealed part
21: a non-sealed part, 21A: a first non-sealed side part, 21B: a second non-sealed side part

1. An absorbent article having a front-rear direction and a width direction, and comprising a top sheet, a back sheet and an absorbent body provided therebetween, wherein
the absorbent body has a high-rigidity part and a low-rigidity part,
the high-rigidity part and the low-rigidity part extend in the front-rear direction, respectively, and are arranged alternately in the width direction,
an anti-slipping member is provided on a non-skin facing side of the back sheet, and
the anti-slipping member is provided so as to astride a first high-rigidity side part, that is the high-rigidity part closest to one end in the width direction of the absorbent body, and a second high-rigidity side part, that is the high-rigidity part closest to the other end in the width direction of the absorbent body, and not to extend outward in the width direction from the first high-rigidity side part and the second high-rigidity side part.

2. The absorbent article according to claim 1, wherein
the absorbent body is configured to contain an absorbent polymer but not contain a pulp fiber between sheet members,
the absorbent body has a sealed part in which the sheet members are bonded to each other and a non-sealed part in which the sheet members are not bonded to each other, and the absorbent polymer is disposed in the non-sealed part, and
the sealed part forms the low-rigidity part and the non-sealed part forms the high-rigidity part.

3. An absorbent article having a front-rear direction and a width direction, and comprising a top sheet, a back sheet and an absorbent body provided therebetween, wherein
the absorbent body is configured to contain an absorbent polymer but not contain a pulp fiber between sheet members,
the absorbent body has a sealed part in which the sheet members are bonded to each other and a non-sealed part in which the sheet members are not bonded to each other, and the absorbent polymer is disposed in the non-sealed part,
the sealed part and the non-sealed part extend in the front-rear direction, respectively, and are arranged alternately in the width direction,
an anti-slipping member is provided on a non-skin facing side of the back sheet, and
the anti-slipping member is provided so as to astride a first non-sealed side part, that is the non-sealed part closest to one end in the width direction of the absorbent body, and a second non-sealed side part, that is the non-sealed part closest to the other end in the width direction of the absorbent body, and not to extend outward in the width direction from the first non-sealed side part and the second non-sealed side part.

4. The absorbent article according to claim 1, wherein
a plurality of the anti-slipping members are aligned in the front-rear direction.

5. The absorbent article according to claim 1, wherein
the anti-slipping member is not provided on a front side of a front end of the absorbent body and on a rear side of a rear end of the absorbent body.

6. The absorbent article according to claim 1, wherein
attaching-fixing members made of a hook member or an adhesive layer are provided on the non-skin facing side of the back sheet on a front side and a rear side of the anti-slipping member.

7. The absorbent article according to claim 6, wherein
the attaching-fixing member is provided so as to overlap with a front end of the absorbent body or on a front side of a front end of the absorbent body, or provided so as to overlap with a rear end of the absorbent body or on a rear side of a rear end of the absorbent body.

8. The absorbent article according to claim 6, wherein
the attaching-fixing member is wider in the width direction than the anti-slipping member.

9. The absorbent article according to claim 1, wherein
the anti-slipping member is formed of a resin layer.

10. The absorbent article according to claim 1, wherein
a fiber sheet is provided between the absorbent body and the back sheet,
the fiber sheet has a larger mass per unit area than the top sheet and the back sheet, and
the fiber sheet is provided so as to overlap with the entire low-rigidity part or the entire sealed part.

11. The absorbent article according to claim 1, wherein
a length of the high-rigidity part or the non-sealed part in the width direction is longer than a length of the low-rigidity part or the sealed part in the width direction.

12. The absorbent article according to claim 1, wherein
the anti-slipping member is formed so as to rise from a surface of the non-skin facing side of the back sheet.

13. The absorbent article according to claim 1, wherein
the anti-slipping member has a durometer hardness A of 20 or more and 90 or less.

14. The absorbent article according to claim 3, wherein
a plurality of the anti-slipping members are aligned in the front-rear direction.

15. The absorbent article according to claim 3, wherein
the anti-slipping member is not provided on a front side of a front end of the absorbent body and on a rear side of a rear end of the absorbent body.

16. The absorbent article according to claim 3, wherein
attaching-fixing members made of a hook member or an adhesive layer are provided on the non-skin facing side of the back sheet on a front side and a rear side of the anti-slipping member.

17. The absorbent article according to claim 16, wherein
the attaching-fixing member is provided so as to overlap with a front end of the absorbent body or on a front side of a front end of the absorbent body, or provided so as to overlap with a rear end of the absorbent body or on a rear side of a rear end of the absorbent body.

18. The absorbent article according to claim 16, wherein
the attaching-fixing member is wider in the width direction than the anti-slipping member.

19. The absorbent article according to claim 3, wherein
the anti-slipping member is formed of a resin layer.

20. The absorbent article according to claim 3, wherein
a length of the high-rigidity part or the non-sealed part in the width direction is longer than a length of the low-rigidity part or the sealed part in the width direction.
